(19) United States
(12) Patent Application Publication
(10) Pub. No.: US 2022/0313069 A1
(43) Pub. Date: Oct. 6, 2022

(54) Systems and Methods for Application of Rigid and Flexible Adhesives for Cable Attachment to BGAs

(71) Applicant: High Speed Interconnects, LLC, Scottsdale, AZ (US)

(72) Inventors: Antonio De La Rosa, Scottsdale, AZ (US); Todd Albertson, Portland, OR (US)

(21) Appl. No.: 17/845,006

(22) Filed: Jun. 21, 2022

Related U.S. Application Data
(63) Continuation-in-part of application No. 16/984,649, filed on Aug. 4, 2020.
(60) Provisional application No. 63/212,792, filed on Jun. 21, 2021.
(60) Provisional application No. 62/885,548, filed on Aug. 12, 2019.

(51) Int. Cl. A61B 1/00; H01R 12/65; H01R 13/405; H01R 13/52; H01B 7/20; H01B 7/02; H01B 3/44; H01B 13/06; H01B 13/004

(52) U.S. Cl. CPC A61B 1/00124; H01R 12/65; H01R 13/405; H01R 13/5224; H01B 7/20; H01B 7/0216; H01B 3/447; H01B 13/067; H01B 13/004

(57) ABSTRACT

A cable assembly, comprising a component, a cable, a rigid adhesive and a flexible adhesive. The cable may be coupled to the component. The rigid adhesive may be applied to cover the connection of the cable to the component. The flexible adhesive may be applied to cover the rigid adhesive. The component may comprise a ball grid array (BGA) and the cable may be coupled to the BGA. A hypodermic tubing may be applied over a portion of the rigid adhesive.

### CROSS-REFERENCE TO RELATED APPLICATIONS

The present application claims the benefit of U.S. Provisional Application Ser. No. 63/212,792, filed Jun. 21, 2021, entitled “Systems and Methods for Application of Rigid and Flexible Adhesives for Cable Attachment to BGAs”; and is a continuation in part of U.S. Patent Application Ser. No. 16/984,649, filed Aug. 4, 2020, entitled “Methods and Apparatus for RF Shield and Cable Attachment System” which claims the benefit of U.S. Provisional Application Ser. No. 62/885,548, filed Aug. 12, 2019, entitled “Methods and Apparatus for Shield and Cable Attachment System” and incorporates the disclosure of each by reference.

### BACKGROUND OF THE TECHNOLOGY

Micro sensing components such as endoscopes, guidewires, catheters, cameras, and the like require small form factors that inherently create installation and manufacturing concerns for discrete cable or coaxial cable termination. For example, devices such as endoscopes, guidewires, catheters, or cameras typically have a ball grid array to facilitate the signal transfers to or from the device and/or to provide power. However, because these types of devices have size constraints on the order of 3 millimeters or less, cable design and proper termination of the cable at the device can be problematic.

Common methods of terminating shielded or coaxial cables to ball grid arrays (BGA) utilize direct connection to the BGA. These types of connections may create a manufacturing concern since connecting such a small length of wire within the space constraints of the assembly requires precision slowing down the manufacturing process and potentially leading to excessive manufacturing efficiencies or assembly quality problems. These types of connections require adhesives to cover the terminations of the cables to the BGA. Further, the terminations of the endoscopic assemblies can be damaged if they are flexed during installation or in use.

### SUMMARY OF THE TECHNOLOGY

Methods and apparatus for application of a combination of rigid and flexible adhesives to cover the terminations of a cable attachment system according to various aspects of the present technology include providing a rigid adhesive in a small quantity to cover the direct termination of the cable to the BGA and then a flexible adhesive is applied to add strain relief and flexibility to the cable assembly.

### BRIEF DESCRIPTION OF THE DRAWINGS

A more complete understanding of the present technology may be derived by referring to the detailed description and claims when considered in connection with the following illustrative figures. In the following figures, like reference numbers refer to similar elements and steps throughout the figures.

FIG. 1 representatively illustrates a perspective view of a component with a BGA with a cable attached thereto utilizing rigid and flexible adhesives in accordance with an exemplary embodiment of the present technology;

FIG. 2A representatively illustrates a side, cross-section view of FIG. 1 of a component with a BGA with a cable attached thereto utilizing rigid and flexible adhesives in accordance with an exemplary embodiment of the present technology;

FIG. 2B representatively illustrates another embodiment of a side, cross-section view taken along the line K-K of a component with a BGA with a cable attached thereto utilizing rigid and flexible adhesives in accordance with an exemplary embodiment of the present technology

FIG. 3A representatively illustrates an end view of the component of FIGS. 1 and 2 installed in a distal end of an endoscope in accordance with an exemplary embodiment of the present technology;

FIG. 3B representatively illustrates a perspective, cross section view taken along the line G-G of FIG. 3A of the component of FIGS. 1 and 2 installed in a distal end of an endoscope in accordance with an exemplary embodiment of the present technology;

FIG. 3C representatively illustrates a side, cross section view taken along the line G-G of FIG. 3A of the component of FIGS. 1 and 2 installed in a distal end of an endoscope in accordance with an exemplary embodiment of the present technology;

FIG. 4A representatively illustrates a perspective view of an additional embodiment of a component with a cable attached thereto utilizing rigid and flexible adhesives in accordance with an exemplary embodiment of the present technology;

FIG. 4B representatively illustrates an end view of the additional embodiment shown in FIG. 4A of a component with a cable attached thereto utilizing rigid and flexible adhesives in accordance with an exemplary embodiment of the present technology;

FIG. 4C representatively illustrates a side, cross-section view of the additional embodiment shown in FIG. 4A taken along the line H-H of FIG. 4B of a component with a cable attached thereto utilizing rigid and flexible adhesives in accordance with an exemplary embodiment of the present technology;

FIG. 4D representatively illustrates a side view of the additional of a component with a cable attached thereto utilizing rigid and flexible adhesives in accordance with an exemplary embodiment of the present technology;

FIG. 5A representatively illustrates a perspective view of an additional embodiment of a component with a cable attached thereto utilizing rigid and flexible adhesives in accordance with an exemplary embodiment of the present technology;

FIG. 5B representatively illustrates an end view of the additional embodiment shown in FIG. 5A of a component with a cable attached thereto utilizing rigid and flexible adhesives in accordance with an exemplary embodiment of the present technology;

FIG. 5C representatively illustrates a side, cross-section view of the additional embodiment shown in FIG. 5A taken along the line J-J of FIG. 5B of a component with a cable attached thereto utilizing rigid and flexible adhesives in accordance with an exemplary embodiment of the present technology; and

FIG. 5D representatively illustrates a side view of the additional with dashed lines to show the internal portions of a component with a cable attached thereto utilizing rigid and flexible adhesives in accordance with an exemplary embodiment of the present technology.

Elements and steps in the figures are illustrated for simplicity and clarity and have not necessarily been rendered according to any particular sequence. For example, steps that may be performed concurrently or in a different order are illustrated in the figures to help to improve understanding of embodiments of the present technology.

### DETAILED DESCRIPTION OF EXEMPLARY EMBODIMENTS

The present technology may be described in terms of functional block components and various processing steps. Such functional blocks may be realized by any number of hardware or software components configured to perform the specified functions and achieve the various results. For example, the present technology may employ various cables, sensors, dielectrics, connection types, circuit cards/boards, and the like, which may carry out a variety of analog or digital (MIPI) functions. In addition, the present technology may be practiced in conjunction with any number of applications, and the system described is merely one exemplary application for the technology. Further, the present technology may employ any number of conventional techniques for providing analog or digital (MIPI) control signals, reducing noise, cross-talk, attenuation, impedance, controlling power, and the like.

Methods and apparatus for a cable attachment system according to various aspects of the present technology may operate in conjunction with any suitable electronic sensor, video system, data collection system, and/or other electronic device. Various representative implementations of the present technology may be applied to any appropriate system for imaging such as a temporarily insertable camera system or any other suitable technology.

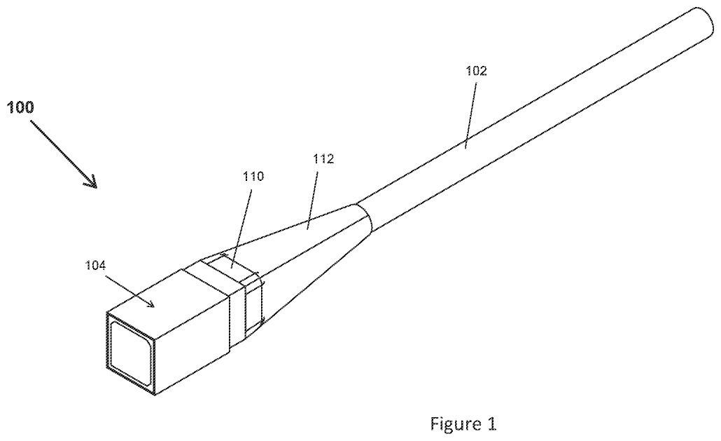

Referring now to FIG. 1, a cable attachment system and apparatus 100 according to various aspects of the present technology may be used to connect one or more coaxial cables to various components 104 within micro sensing components such as endoscopes, guidewires, catheters, cameras, and the like. The cable attachment system and apparatus 100 may comprise a cable assembly 102, a component 104, a rigid adhesive 110 and a flexible adhesive 112.

Individual coaxial cables within the cable assembly 102 are used to transmit signals from a source device at a source end to a receiving end such as a display or memory device. It should be understood that there can be a single cable in the cable assembly 102 or multiple cables. The signals may comprise any suitable electrical signals, for example a combination of data, control signals, and power. For example, in one embodiment for use with an imaging system using a camera, a first coaxial cable may provide electrical power to the camera. The camera may be configured with an integrated circuit such as a CMOS imaging sensor, an array, a BGA, or other sensing device that is connected directly to the flex circuit and cable attachment system 100. A second coaxial cable may transmit a clock signal between the camera and the receiving end of the RF shield and cable attachment system 100. A third coaxial cable may transmit an image signal from the camera to the receiving end where the image signal may be displayed or analyzed for processing. In alternative embodiments, additional coaxial cables may be included to transmit additional power lines or signals as required. Similarly, fewer cables may be used if the particular application requires the transmission of fewer signals or less power lines through the RF shield and cable attachment system 100.

The coaxial cables 102 may comprise any suitable type of coaxial cable, such as a cable comprising a center conductor, a dielectric surrounding the center conductor, and a shield covering the dielectric.

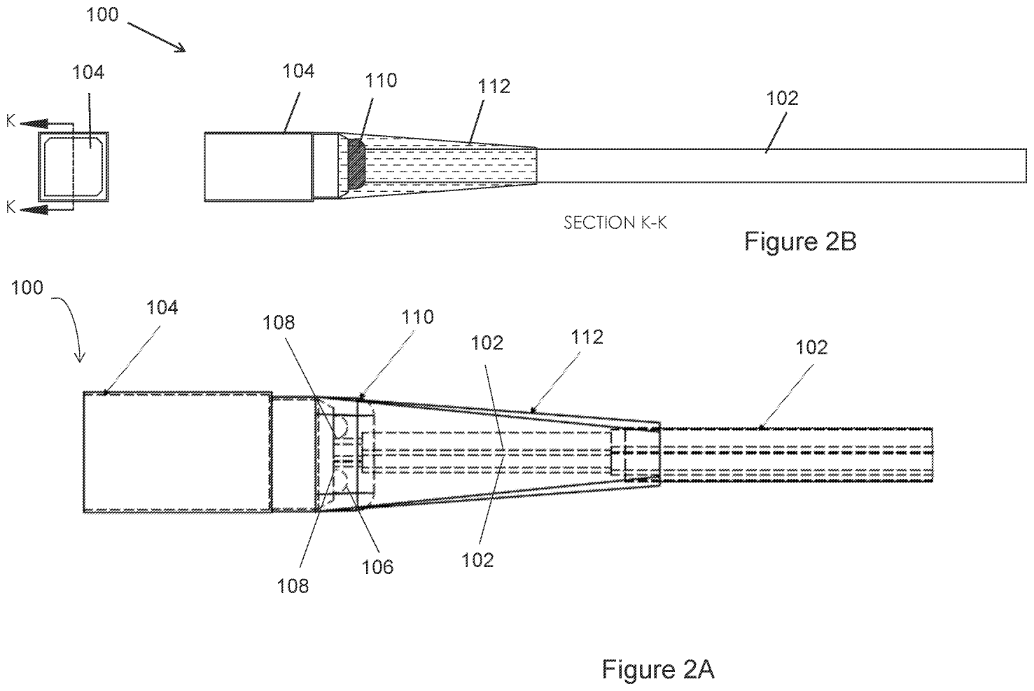

In various embodiments, referring to FIGS. 1 and 2, the cables 102 may be coupled to a BGA 106 on the component 104 at a termination interconnect 108. The termination interconnect 108 is where the cables 102 are connected to the component 104 or to a BGA 106. Once connected, the termination interconnect 108 may be covered or encased by a thin layer of rigid adhesive 110. The rigid adhesive 110 may be applied with a minimum amount necessary to cover the terminations of the cables 102 as close to the component 104 or BGA 106 as possible. The rigid adhesive 110 may be applied over cables 102 attached to electrical components 104 with BGAs 106 or other conductive pads, cable 102 attached to PCBs and flex circuits, cable 102 attached to connectors, and the like. Once the rigid adhesive 110 is applied over the termination interconnect 108 where the cables 102 are attached to the component 104 or BGA 106, a flexible adhesive 112 may be applied over the rigid adhesive 110 and a portion of the cable 102. The application of the flexible adhesive 112 adds strain relief while also allowing the cable attachment system 100 to flex during use and installation.

The rigid adhesive 110 may be formed around the termination interconnect 108 and then the flexible adhesive 112 may be formed around the rigid adhesive 110 and cable 102. In one embodiment, the rigid adhesive 110 may be formed around the termination interconnect 108 and then the flexible adhesive 112 may be formed around a portion the rigid adhesive 110. In one embodiment, the rigid adhesive 110 may be formed around the termination interconnect 108 and then the flexible adhesive 112 may be formed around all of the rigid adhesive 110 and a portion of the cable 102. In one embodiment, the rigid adhesive 110 may be formed around the termination interconnect 108 and then the flexible adhesive 112 may be formed around a portion the rigid adhesive 110 and a portion of the cable 102.

In various embodiments, the rigid adhesive 110 may be opaque to prevent light from affecting an image sensor. In various embodiments, the rigid adhesive 110 may be black opaque. In various embodiments, the rigid adhesive 110 may comprise one of cyanoacrylates, acrylic adhesives, 2 part epoxies, silicone adhesives, UV curable adhesives, and urethane adhesives. The rigid adhesives 110 may comprise a material hard enough to prevent stress from damaging the cable 102 and termination interconnect 108. In one embodiment a 2 part epoxy is used for the opaque adhesive. For example, in one embodiment, the rigid adhesive 110 may comprise a material having a Shore hardness of about 89D.

In various embodiments, the flexible adhesive 112 may comprise one of cyanoacrylates, acrylic adhesives, 2 part epoxies, silicone adhesives, UV curable adhesives, and urethane adhesives. In one embodiment, the flexible adhesive 112 may comprise an acrylic adhesive. For example, in one embodiment, the flexible adhesive 112 may comprise a material having a Shore hardness of about 77A. In one embodiment, a pre-molded flexible boot or heat shrunk tubing may be used in lieu of the flexible adhesive 112. In one embodiment, the rigid adhesive 110 may be removed and only a flexible adhesive 112 used to enclose the termination interconnect 108 and the cables 102.

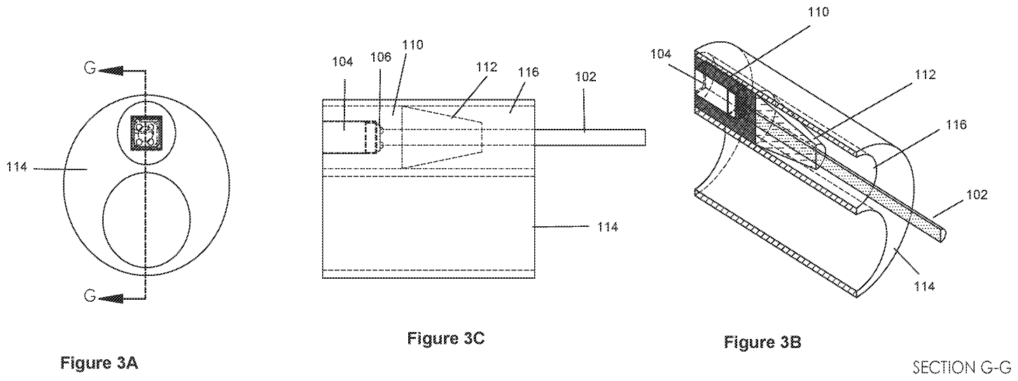

Referring now to FIGS. 3A-3C, the cable system 100 may be used in conjunction with an endoscope tip or housing 114. As shown in the figures the cable system 100 may be received within a sleeve 116 within the endoscope tip or housing 114. The endoscope tip or housing 114 may comprise one of stainless steel, PEEK, or a liquid crystal polymer (LCP) material.

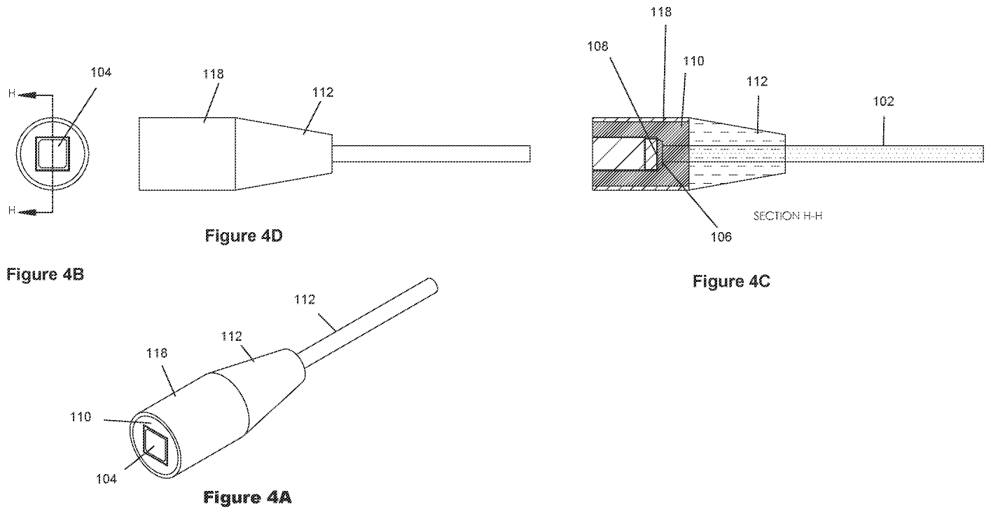

Referring now to FIGS. 4A-4D, the cable system 100 may comprise a hypodermic tubing 118 applied over the rigid adhesive 110. The hypodermic tubing 118 material may comprise a stainless steel material. The hypodermic tubing 118 may be applied over the rigid adhesive 110 or over the flexible adhesive 112 when the flexible adhesive 112 is applied over the rigid adhesive 110.

In one embodiment, the component 104 and or the BGA 106 component connect to the cable 102 may be placed inside the hypodermic tubing 118. The cavity within the hypodermic tubing 118 may be filled with the rigid adhesive 110 to cover the connection between the component 104 and the cable 102 or the BGA 106 and the cable 102. The rigid adhesive 110 may then be allowed to cure before the flexible adhesive 112 may be applied over the rigid adhesive 110.

In one embodiment, the rigid adhesive 110 may be applied over the component 104 and or the BGA 106 component connection to the cable 102. The hypodermic tubing 118 may be placed over the component 104, and then the flexible adhesive 112 may be applied to adhere everything together.

As discussed above, in one embodiment, the rigid adhesive 110 may be formed around the termination interconnect 108 and then the flexible adhesive 112 may be formed around the rigid adhesive 110 and cable 102. The hypodermic tubing 118 may be applied over the rigid adhesive 110 or over the flexible adhesive 112 when the flexible adhesive 112 is applied over the rigid adhesive 110

In one embodiment, the rigid adhesive 110 may be formed around the termination interconnect 108 and then the flexible adhesive 112 may be formed around a portion the rigid adhesive 110. The hypodermic tubing 118 may be applied over the rigid adhesive 110 or over the flexible adhesive 112 when the flexible adhesive 112 is applied over the rigid adhesive 110.

In one embodiment, the rigid adhesive 110 may be formed around the termination interconnect 108 and then the flexible adhesive 112 may be formed around all of the rigid adhesive 110 and a portion of the cable 102. The hypodermic tubing 118 may be applied over the rigid adhesive 110 or over the flexible adhesive 112 when the flexible adhesive 112 is applied over the rigid adhesive 110

In one embodiment, the rigid adhesive 110 may be formed around the termination interconnect 108 and then the flexible adhesive 112 may be formed around a portion the rigid adhesive 110 and a portion of the cable 102. The hypodermic tubing 118 may be applied over the rigid adhesive 110 or over the flexible adhesive 112 when the flexible adhesive 112 is applied over the rigid adhesive 110.

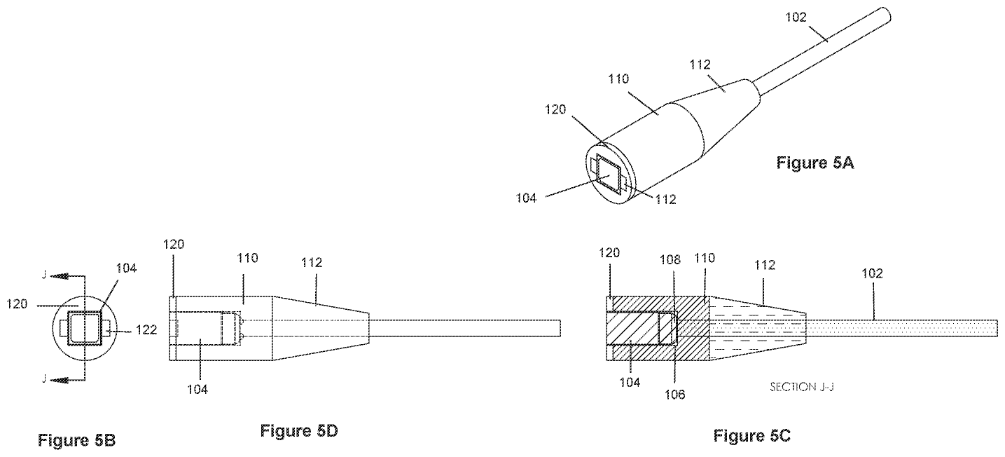

Referring now to FIGS. 5A-5D, the cable system 100 may comprise an organizer tip 120. The organizer tip 120 may be coupled to and surround an end of the component 104. The organizer tip 120 may comprise a space to accommodate an LED 122.

In operation, the rigid and flexible adhesives 110, 112 may be applied over cables 102 attached to electrical components 104 with BGAs 106 or other conductive pads, cable 104 attached to PCBs and flex circuits, cable 104 attached to connectors. In various embodiments, the rigid and flexible adhesives 110, 112 may be applied by hand, using a pneumatic dispensing machines or pin transfer techniques. The rigid and flexible adhesives 110, 112 could also be applied by a robot using the same methods. In various embodiment, the rigid and flexible adhesives 110, 112 could be applied by 2 step injection molding; first a low temp/low pressure rigid adhesive 110 followed by the flexible adhesive 112. In various embodiments, the components of the cable assembly 100 may be placed in a cavity and the rigid and flexible adhesives 110, 112 may be applied in the manner discussed above.

In one embodiment, the BGA 106 component may be placed inside the hypodermic tubing 118 and then the cavity within the hypodermic tubing 118 may be filled with the rigid adhesive 110 to cover the connection between the component 104 and the cable 102 or the BGA 106 and the cable 102. The rigid adhesive 110 may then be allowed to cure before the flexible adhesive 112 may be applied over the rigid adhesive 110.

In one embodiment, the rigid adhesive 110 may be applied over the BGA 106, the hypodermic tubing 118 may be placed over the component 104, and then the flexible adhesive 112 may be applied to adhere everything together.

This method of connecting the coaxial cables 102 to the BGA 106 of a component 104 may provide an improved ability for flexing of the cable assembly 100. For example, camera systems that are used to view inside the human body often require that the camera system be able to bend and adjust according to a component inner diameter used to penetrate or diagnose a human body, such as a scope, catheter, guidewire, or flexible introducer sheath.

These and other embodiments for methods of forming the cable attachment system 100 may incorporate concepts, embodiments, and configurations as described above. The particular implementations shown and described are illustrative of the technology and its best mode and are not intended to otherwise limit the scope of the present technology in any way. Indeed, for the sake of brevity, conventional manufacturing, connection, preparation, and other functional aspects of the system may not be described in detail. Furthermore, the connecting lines shown in the various figures are intended to represent exemplary functional relationships and/or physical couplings between the various elements. Many alternative or additional functional relationships or physical connections may be present in a practical system.

The technology has been described with reference to specific exemplary embodiments. Various modifications and changes, however, may be made without departing from the scope of the present technology. The description and figures are to be regarded in an illustrative manner, rather than a restrictive one and all such modifications are intended to be included within the scope of the present technology. Accordingly, the scope of the technology should be determined by the generic embodiments described and their legal equivalents rather than by merely the specific examples described above. For example, the steps recited in any method or process embodiment may be executed in any order, unless otherwise expressly specified, and are not limited to the explicit order presented in the specific examples. Additionally, the components and/or elements recited in any apparatus embodiment may be assembled or otherwise operationally configured in a variety of permutations to produce substantially the same result as the present technology and are accordingly not limited to the specific configuration recited in the specific examples.

Benefits, other advantages and solutions to problems have been described above with regard to particular embodiments; however, any benefit, advantage, solution to problems or any element that may cause any particular benefit, advantage or solution to occur or to become more pronounced are not to be construed as critical, required or essential features or components.

As used herein, the terms “comprises”, “comprising”, or any variation thereof, are intended to reference a non-exclusive inclusion, such that a process, method, article, composition or apparatus that comprises a list of elements does not include only those elements recited, but may also include other elements not expressly listed or inherent to such process, method, article, composition or apparatus. Other combinations and/or modifications of the above-described structures, arrangements, applications, proportions, elements, materials or components used in the practice of the present technology, in addition to those not specifically recited, may be varied or otherwise particularly adapted to specific environments, manufacturing specifications, design parameters or other operating requirements without departing from the general principles of the same.

The present technology has been described above with reference to an exemplary embodiment. However, changes and modifications may be made to the exemplary embodiment without departing from the scope of the present technology. These and other changes or modifications are intended to be included within the scope of the present technology, as expressed in the following claims.

What is claimed:

1. A cable assembly, comprising:
a component;
a cable coupled to the component at a termination interconnect;
a rigid adhesive covering the termination interconnect; and
a flexible adhesive covering a portion of the rigid adhesive.

2. The cable assembly of claim 1, wherein the component comprises a ball grid array (BGA).

3. The cable assembly of claim 2, wherein the cable is coupled to the BGA at the termination interconnect.

4. The cable assembly of claim 1, further comprising a hypodermic tubing applied over a portion of the rigid adhesive.

5. The cable assembly of claim 1, further comprising a hypodermic tubing applied over all of the rigid adhesive.

6. The cable assembly of claim 1, further comprising a hypodermic tubing applied over a portion of the rigid adhesive and the flexible adhesive.

7. The cable assembly of claim 1, further comprising a hypodermic tubing applied over all of the rigid adhesive and the flexible adhesive.

8. The cable assembly of claim 1, wherein the rigid adhesive comprises at least one of cyanoacrylates, acrylic adhesives, 2 part epoxies, silicone adhesives, UV curable adhesives, and urethane adhesives.

9. The cable assembly of claim 8, wherein the rigid adhesive comprises a black opaque adhesive.

10. The cable assembly of claim 1, wherein the flexible adhesive comprises at least one of cyanoacrylates, acrylic adhesives, 2 part epoxies, silicone adhesives, UV curable adhesives, and urethane adhesives.

11. The cable assembly of claim 1, wherein the rigid adhesive comprises an acrylic adhesive.

12. A method of assembling a cable assembly, comprising:
coupling a cable to a component at a termination interconnect;
applying a rigid adhesive to cover the termination interconnect; and
applying a flexible adhesive to cover a portion of the rigid adhesive.

13. The method of claim 12, further comprising applying a hypodermic tubing over the rigid and flexible adhesives.

14. The method of claim 12, wherein the component comprises a ball grid array (BGA) and the cable is coupled to the BGA at the termination interconnect.

15. The method of claim 12, wherein the flexible adhesive is applied to cover all of the rigid adhesive and a portion of the cable.

16. A method of assembling a cable assembly comprising:
coupling a cable to a component at a termination interconnect;
applying a hypodermic tubing over the termination interconnect;
applying a rigid adhesive to cover the termination interconnect within the hypodermic tubing; and
applying a flexible adhesive to cover the hypodermic tubing and the cable.

17. The method of claim 16, wherein the component comprises a ball grid array (BGA) and the cable is coupled to the BGA.

18. The method of claim 16, wherein the flexible adhesive is applied to cover all of the rigid adhesive and a portion of the cable.
